(19) United States
(12) Patent Application Publication
(10) Pub. No.: US 2012/0108559 A1
(43) Pub. Date: May 3, 2012

(54) RECEPTOR ACTIVATOR OF THE RANK RECEPTOR COMPLEX INHIBITOR

(75) Inventors: Ramachandran Murali, Beverly Hills, CA (US); Mark I. Greene, Penn Valley, PA (US)

(73) Assignee: THE TRUSTEES OF THE UNIVERSITY OF PENNSYLVANIA, Philadelphia, PA (US)

(21) Appl. No.: 13/144,568

(22) PCT Filed: Jan. 7, 2010
(86) PCT No.: PCT/US10/20303

§ 371 Date: Sep. 29, 2011

Related U.S. Application Data
(60) Provisional application No. 61/144,589, filed on Jan. 14, 2009.

(51) Int. Cl. A61K 31/585; C07J 17/00; A61P 19/08

(52) U.S. Cl. 514/175; 540/105

(57) ABSTRACT

Methods and pharmaceutical compositions utilizing compounds of Formula II: for inhibiting osteoclastogenesis and the activity of osteoclasts in patients.

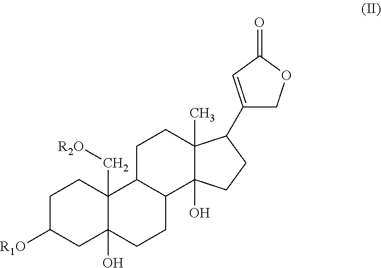

### CROSS REFERENCE TO RELATED APPLICATIONS

This application claims the benefit of priority to U.S. Provisional Patent Application Ser. No. 61/144,589, filed Jan. 14, 2009, which is herein incorporated by reference in its entirety.

### TECHNICAL FIELD

The present invention relates to the methods of down-modulating osteoclastogenesis activity, thereby inhibiting bone matrix erosion and thus preventing bone loss and treating bone diseases.

### BACKGROUND

Bone diseases such a osteoporosis and Paget's disease are characterized by loss of bone. In many cases, bone loss leads to fractures in patients. In addition to the pain and suffering, patients become physically impaired, which lead to further negative consequences to the patients' health and quality of life. Moreover, the associated economic costs are tremendous.

Receptors and ligands of the Tumor Necrosis Factor (TNF) family have recently been shown to play an essential part in the differentiation and activity of osteoclasts.

RANKL (for Receptor Activator of NF (Necrosis Factor)-κB Ligand; also called TRANCE for TNF (Tumor Necrosis Factor)-Related, Activation induced Cytokine, or ODF, for osteoclast differentiation factor) is a TNF family ligand expressed on the surface of stromal/osteoblast cells. The receptor for RANKL is RANK (Receptor Activator of NF-κB), which is located on the surface of osteoblast precursors and mature osteoclasts. The RANK/RANKL interaction induces osteoclast development (rapid differentiation in bone marrow of osteoclast precursors into mature osteoclasts) as well as activation of mature osteoclasts (increased functional activity and reduced apoptosis). The effects of RANK and RANKL on bone resorption have been extensively documented in vitro and in vivo.

These features have been recently discussed in U.S. Pat. No. 6,673,771 which is incorporated herein by reference in its entirety for all purposes.

### SUMMARY

The present invention relates to methods of inhibiting osteoclastogenesis and the bone resorbing activity of mature osteoclasts. According to the present invention, an amount of a TRANCE/RANK inhibitor effective to inhibit osteoclast bone erosion activity is administered to a patient.

The present invention relates to methods of treating patients who have diseases characterized by bone loss. According to the present invention, an amount of a TRANCE/RANK inhibitor effective to inhibit osteoclastogenesis is administered to a patient.

The present invention also relates to pharmaceutical compositions which comprise a TRACE/RANK inhibitor in an effective amount to inhibit osteoclastogenesis.

The present invention further relates to methods of modulating dendritic cell maturation, T cell proliferation, and/or CD40 receptor systems in an individual comprising the step of administering to the individual an amount of a TRANCE/RANK inhibitor effective to modulate dendritic cell maturation, T cell proliferation, and/or CD40 receptor systems.

### BRIEF DESCRIPTION OF THE FIGURES

FIG. 1 shows the response of in vitro testing using Compounds III-1 and II-A-1 as a function of concentration.

FIG. 2 shows the response of in vivo testing on bone density of varying concentrations of Compound II-A-1.

### DETAILED DESCRIPTION OF ILLUSTRATIVE EMBODIMENTS

As used herein, the term “TRANCE/RANK inhibitors” refers to the compounds which inhibit osteoclastogenesis and/or osteoclast function, including those described in PCT application Ser. No. PCT/US99/15062 filed Jul. 1, 1999 and U.S. Pat. No. 6,673,771 issued Jan. 6, 2004.

As used herein, the term “diseases characterized by bone loss” is meant to refer to diseases, conditions, disorders, and syndromes which have as a symptom or pathology a decrease in bone mass or density. Examples of diseases characterized by bone loss include osteoporosis, Paget's disease, metastatic bone disease, rheumatoid arthritis and periodontal bone disease.

As used herein, the term “therapeutically effective amount” is meant to refer to an amount of a compound which produces a medicinal effect observed as a reduction in the rate of bone loss in an individual when that amount of a compound is administered to an individual who is susceptible to or suffering from a disease characterized by bone loss. Therapeutically effective amounts are typically determined by the effect they have compared to the effect observed when a composition which includes no active ingredient is administered to a similarly situated individual.

The present invention relates to methods of inhibiting osteoclastogenesis and the bone resorbing activity of mature osteoclasts. According to the present invention, an amount of a TRANCE/RANK inhibitor effective to inhibit osteoclast bone erosion activity is administered to a patient.

The present invention also relates to methods of treating patients who have diseases characterized by bone loss. According to the present invention, an amount of a TRANCE/RANK inhibitor effective to inhibit osteoclastogenesis is administered to a patient.

The present invention further relates to pharmaceutical compositions which comprise a TRACE/RANK inhibitor in an effective amount to inhibit osteoclastogenesis.

The present invention relates to methods of modulating dendritic cell maturation, T cell proliferation, and/or CD40 receptor systems in an individual comprising the step of administering to the individual an amount of a TRANCE/RANK inhibitor effective to modulate dendritic cell maturation, T cell proliferation, and/or CD40 receptor systems.

Some of the compounds described in U.S. Pat. No. 6,673,771 as TRANCE/RANK inhibitors belong to a genus that encompasses compounds having a similar structure to those of the present invention. For example, U.S. Pat. No. 6,673,771 describes compounds of Formula III:

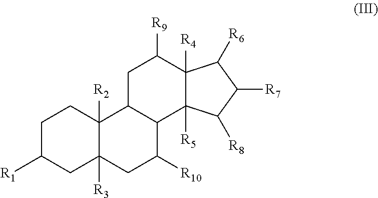

wherein:
R₁═O(NO₂), acetate, hydroxy, acetate, O(CO)(CH₂)₂COOH, O(CO)CH₂Br, O(CO)CH₂Cl, O(CO)CH₂N(CH₃)₃⁺, or O-c-C₅H₉O;
R₂═CH₂O(NO₂), —CHO, cyano, methyl, COOH, CHNOH, CH₂O(CO)(CH₂)₂COOH, CHN(NH)CONH₂, CHN(NH)C₆H₅, CHN(CH₂)C₆H₅, CHN(CH₂)₂OH, CHNC₆H₅, or CHN(NH)CSNH₂;
R₃=hydroxy or hydrogen;
R₄=methyl;
R₅=hydroxy;
R₆=c-C₄H₃O₂, N(NHCO)C₆H₄Cl, N(NHCO)C₆H₄F, COOH, O, (CO)CH₃, CH(CH₃)(CH₂)₂COOH, CH(CH₃)(CH₂)₂COOCH₃, benzoate, or hydroxy;
R₇=hydrogen, O(CO)CH₂N(CH₃)₃⁺, or acetate;
R₈=hydroxy or hydrogen;
R₉═O, hydroxy, or hydrogen; and
R₁₀═O or hydrogen.
   The patent also identifies the following specific compound:

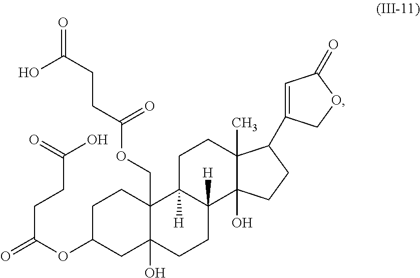

and describes its usefulness as a TRANCE/RANK inhibitor.

Applicants have discovered that a related group of compounds, those of Formula II shown below (where R₁ and R₂ both comprise alkyl or alkanoyl groups) show previously unrecognized and unexpected biological activity in the treatment of osteoclastogenesis. According to some embodiments of the invention, the compound of Formula II-A or II-A-1 is preferred.

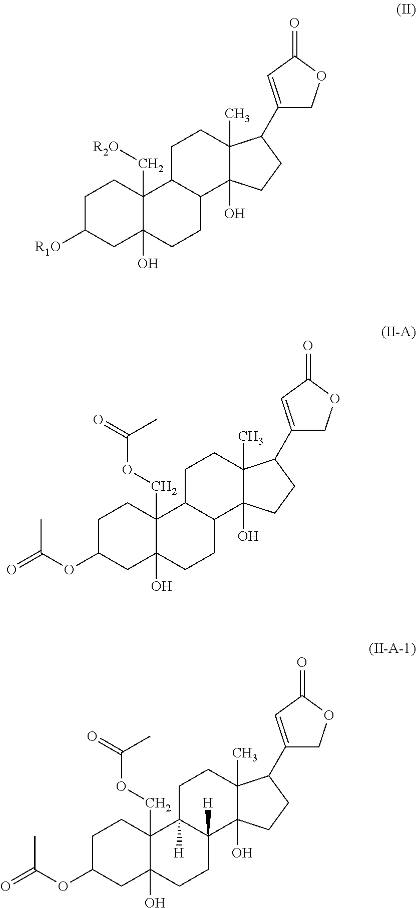

According to the invention, TRANCE/RANK inhibitors useful in the invention to treat diseases characterized by bone loss may be formulated and administered as follows. The compounds of the invention, may be administered to a subject per se or in the form of a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active peptides or peptide analogues into preparations which can be used pharmaceutically. Proper formulation is dependent upon the route of administration chosen.

For topical administration the compounds of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hank's solution, Ringer's solution, or physiological saline buffer. The solution may contain formulatory agents such as suspending, stabilizing and/or dispersing agents.

Alternatively, the compounds may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, 1,1,1,2-tetrafluoroethane, 1,1,1,2,3,3,3-heptafluoropropane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free acids or bases or as pharmaceutically acceptable salts. Pharmaceutically acceptable salts are those salts which substantially retain the antimicrobial activity of the free bases and which are prepared by reaction with inorganic acids. Pharmaceutical salts tend to be more soluble in aqueous and other protic solvents than are the corresponding free base forms.

The compounds of the invention will generally be used in an amount effective to achieve the intended purpose. For use to treat or prevent TNF/TRANCE-associated disorders, the compounds of the invention, or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that inhibits 50% of TRANCE/RANK-binding interactions). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day. Some preferred dosages range from 1 nM to 500 mM. Some preferred dosages range from 1 mM to 500 mM. Some preferred dosages range from 1 mg to 500 mg. Some preferred dosages range from 1000 mg to 3000 mg. Some preferred dosages range from 1500 mg to 2500 mg. According to the invention, TRANCE/RANK inhibitors are administered one to four times per day.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs.

Preferably, a therapeutically effective dose of the compounds described herein will provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use human. The dosage of the compounds described lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in: The Pharmacological Basis of Therapeutics, Ch. 1, p. 1). Preferred dosages range from 1 nM to 500 mM.

Pharmaceutical compositions according to the present invention comprise TRANCE/RANK inhibitors formulated as described above in therapeutically effective doses. In some embodiments, the pharmaceutical compositions is sterile and pyrogen free.

Other aspects of the present invention include the use of TRANCE/RANK inhibitors in methods involving other cell types in which TRANCE/RANK mediated signaling is involved in cell development and/or activity. Such cell types include antigen presenting cells such as dendritic cells and lymphocytes. Anderson et al. 1997 Nature 390:175-179, refer to the RANK/RANKL in T cells and dendritic cells. Similarly, Kong et al. 1999 Immunol. and Cell Biology 77:188-193 refer to osteoprotegerin ligand as a common link between osteoclastogenesis, lymphnode formation and lymphocyte development. In addition, Wong et al. 1999 J. Leukocyte Biology 65:715-724 refer to TRANCE as regulating dendritic cell and osteoclast function. TRANCE/RANK inhibitors formulated as described above in effective doses can be used to modulate dendritic cell maturation and function, T cell proliferation and CD40 receptor systems.

### EXAMPLES

### Example 1

### CIAM Inhibitor Effect on Trance (RANKL)-Induced Osteoclast Formation, In Vitro

The effects of the compound of Formula II-A-1 on TRANCE-mediated osteoclastogenesis were evaluated against murine osteoclast precursors using a TRAP (tartrate resistant acid phosphatase) assay. The techniques comprising this method are described more fully in U.S. Pat. No. 6,673,771.

The compound of Formula II-A-1 was obtained from Sigma-Aldrich Fine Chemicals, Milwaukee, Wis. (Catalog # R-221910). The compound of Formula III-1 was obtained from ChemDiv., Inc. San Diego, Calif. (Catalog #0449-0070).

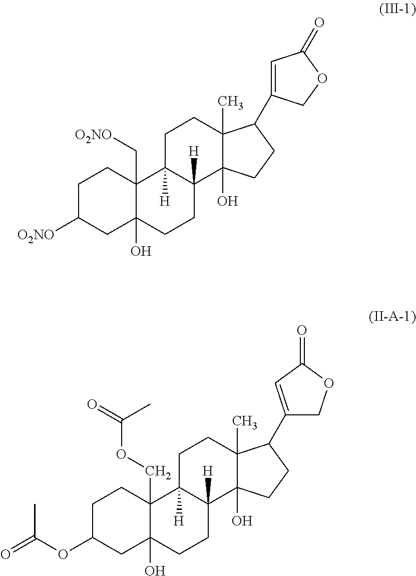

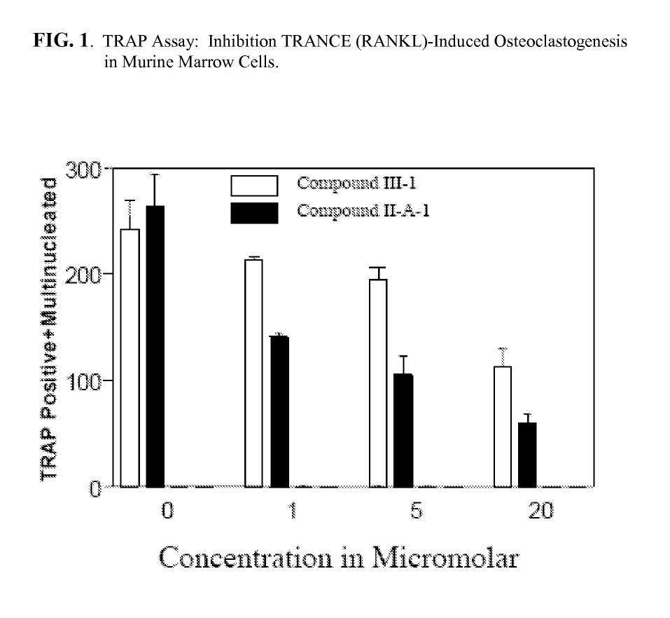

Osteoclast precursors were cultured in 96-well plates (1×105/mL, 200 μL/well) for 3 days in the presence of murine CSF-1 (30 ng/mL) and TRANCE (300 ng/mL) with or without the compounds of Formulae III-1 and II-A-1 at the indicated concentration. After 3 days, the cells were fixed and stained for tartrate-resistant acid phosphatase (TRAP). TRAP-positive multinucleated cells were counted as osteoclast-like multinucleated cells. As shown in FIG. 1, the compounds of Formulae III-1 and II-A-1 inhibited TRANCE induced osteoclast formation in vitro in a dose-dependent manner.

### Example 2

### In Vivo Effects

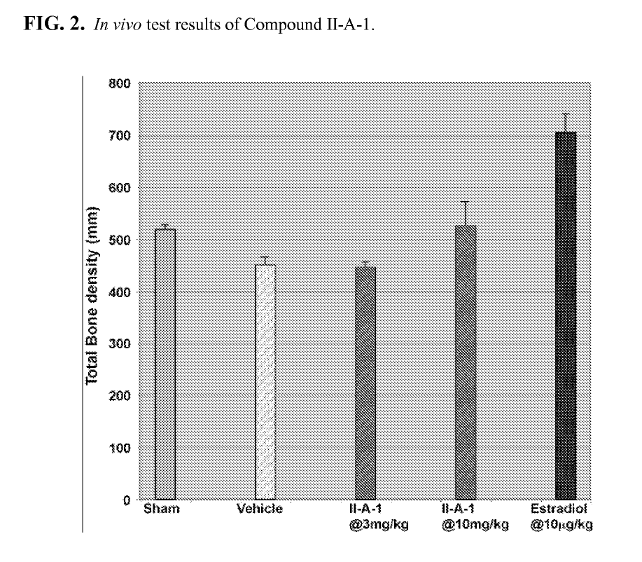

The compound of Formula II-A was tested, in vivo, in the ovarectomized mouse model as described in Cheng, et al., 2004 (Cheng, X., Kinosaki, M., Tamaki, M., Choi, Y., Zhang, H., & Murali, R., J. Biol. Chem., Vol. 279: 8269-827 (2004) which is incorporated by reference herein). This animal model has been validated for human postmenopausal osteoporosis. As shown in FIG. 2, the compound showed a modest and dose-dependent effect at 5-10 μg/mL doses compared to a vehicle control and a sham. Estradiol, administered at a dose of 10 mg/kg, was also tested as a positive control.

What is claimed:

1. The use of a TNF (tumor necrosis factor)-related activation induced cytokine (TRANCE)/receptor activator of NF-κB ligand (RANK) inhibitor for the preparation of a medicament for the inhibition of osteoclastogenesis or osteoclast function or both in a patient having a disease characterized bone loss, wherein the inhibitor is a compound having the Formula II-A:

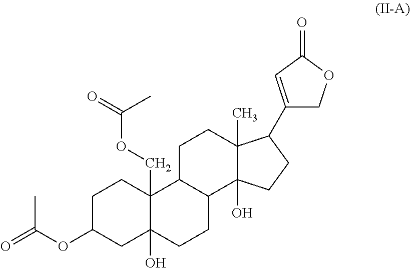

2. The use of claim 1, wherein the compound is

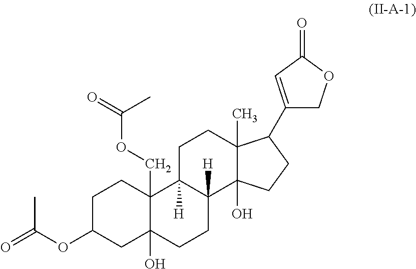

3. The use of a TNF (tumor necrosis factor)-related activation induced cytokine (TRANCE)/receptor activator of NF-κB ligand (RANK) inhibitor to modulate dendritic cell maturation, T cell proliferation, CD40 receptor systems, or two or more of these functions or systems, wherein said TRANCE/RANK inhibitor is a compound having the Formula II-A

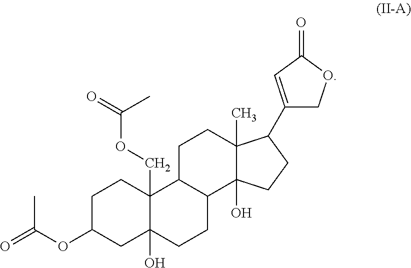

4. The use of claim 3, wherein the compound is

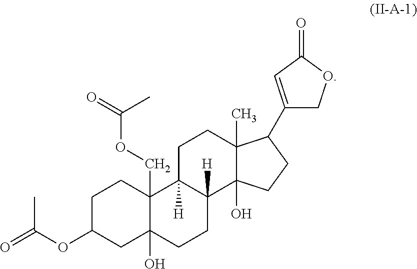

5. A method of treating a patient having a disease characterized by bone loss, comprising the step of administering to said patient a pharmaceutically effective amount of a compound having the Formula II-A

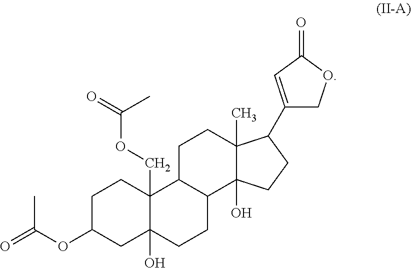

6. The method of claim 5, wherein the compound is

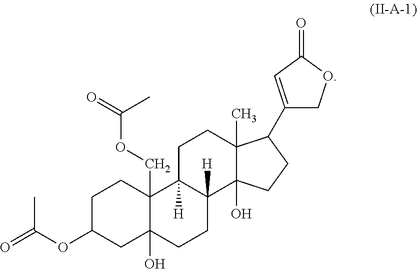

7. A pharmaceutical composition comprising a TRACE/RANK inhibitor in an amount effective to inhibit osteoclastogenesis, wherein said TRANCE/RANK inhibitor is a compound having the Formula II-A

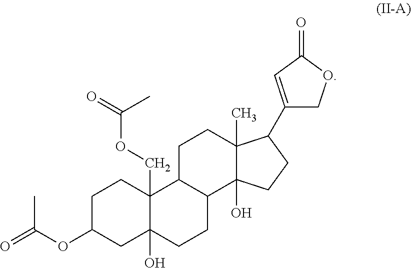

8. The composition of claim 7, wherein the compound is [chemical structure]
